(19) United States
(12) Patent Application Publication
(10) Pub. No.: US 2024/0065979 A1
(43) Pub. Date: Feb. 29, 2024

(54) Protection of Polyunsaturated Fatty Acids, Lipid-Soluble Vitamins, and Lipid-Soluble Medicaments from Oxidation

(71) Applicant: One Idea LLC, Merced, CA (US)

(72) Inventor: Juan P. Russi, Buenos Aires (AR)

(73) Assignee: One Idea LLC, Merced, CA (US)

(21) Appl. No.: 18/259,904

(22) PCT Filed: Dec. 28, 2021
(86) PCT No.: PCT/US21/65358

§ 371 Date: Jun. 29, 2023

Related U.S. Application Data
(60) Provisional application No. 63/199,438, filed on Dec. 29, 2020.

(51) Int. Cl. A61K 9/107; A61K 31/07; A61K 31/122; A61K 31/355; A61K 31/593; A61K 45/06; A61K 47/10; A61K 47/12; A61K 47/24

(52) U.S. Cl. CPC A61K 9/1075; A61K 31/07; A61K 31/122; A61K 31/355; A61K 31/593; A61K 45/06; A61K 47/10; A61K 47/12; A61K 47/24

(57) ABSTRACT

Disclosed are methods of preparing PUFA-, lipid-soluble vitamin-, lipid-soluble medicament-encapsulated, or other lipid-soluble molecules micelles that enhance shelf life and prevent oxidation by inducing a Maillard reaction between a reducing sugar and a lipid emulsifying agent having at least one amino group in the presence of a PUPA, lipid-soluble vitamin or lipid-soluble medicament. Method steps include: mixing a reducing sugar, and a PUPA, lipid-soluble vitamin or lipid-soluble medicament, with a lipid emulsifying agent having at least one amino group and a purity of at least 95 wt %, and heating the resulting micelle mixture for sufficient time at a sufficient temperature and under reduced pressure below 1 atm with sufficient moisture so that a Maillard reaction occurs between the lipid amino groups and the reducing carbohydrate to provide a Maillard reaction product coating on the micelles that is effective to prevent oxidation of the PUPA, lipid-soluble vitamin or lipid-soluble medicament.

### CROSS REFERENCE TO RELATED APPLICATION

This application claims priority under 35 U.S.C. § 119(e) to U.S. Provisional Patent Application Ser. No. 63/199,438, filed on Dec. 29, 2020, which is hereby incorporated by reference in its entirety.

### FIELD OF THE INVENTION

The present invention relates to preventing polyunsaturated fatty acids (PUFA), lipid-soluble vitamins, and lipid-soluble medicaments from being oxidized by using purified amine-containing lipids to encapsulate the PUFA, vitamins, medicaments and other lipid-soluble molecules; and exposing the encapsulated species to a controlled Maillard reaction. The present invention also relates to encapsulated PUFA, vitamins, medicaments and other lipid-soluble molecules; made by the inventive process.

### BACKGROUND

Polyunsaturated fatty acids, especially omega-3 and omega-6 fatty acids, are essential for normal physiological functioning and for the health of animals, including domesticated species and humans. The omega-3 family of fatty acids includes eicosapentaenoic acid (EPA) and docosahexaenoic acid (DHA), which are essential nutrients found to be beneficial on the health of animals and humans.

In the human and mammals, EPA and DHA play key roles in regulating body homeostasis. Additionally, EPA and DHA are precursors to anti-inflammatory molecules such as eicosanoids and resistins. Therefore, they can be protective against inflammatory diseases, cancer, cardiovascular diseases, and other chronic diseases.

Due to their chemical structure, PUFA are sensitive to rancidification, which means that PUFA are easily oxidized, even at room temperature, causing undesirable flavors, odors, colors, and textures; and lowering the nutritional quality and safety of lipid-containing foods. Rancidification of lipids can have a significant effect on food quality even when the lipid content is very small. Thus, oxidative deterioration of PUFA in foods is a serious problem in many sectors of the food industry.

The same is true for lipid-soluble (fat-soluble) vitamins, such as vitamins A, D, E and K. Likewise, undesired oxidation of lipid-soluble medicaments is problematic.

U.S. Pat. No. 8,741,337 discloses the use of Maillard reaction products to coat phospholipid-encapsulated PUFA. However, the encapsulation is hindered by the low reactivity of the phospholipid.

U.S. Pat. No. 8,221,809 discloses the use of the Maillard reaction product to coat different PUFA using extrusion and vacuum drying. However, the process is hindered by immiscibility of the reagents.

Similarly, patent EP2166874 discloses the use of a Maillard reaction product to coat different PUFA with a mixture of proteins and reducing sugars using extrusion and vacuum drying or an extrusion unit that has a vacuum degassing barrel to carry out part of the extrusion at pressures lower than atmospheric pressure (0.4-0.6 ATM)

The Maillard reaction occurs in three stages. In the initial stage, which is reversible, a colorless product, without absorption of ultraviolet light (about 280 nm) is produced through two reactions: sugar-amine condensation and Amadori rearrangement. Should the reaction proceed beyond the Amadori rearrangement to the Strecker degradation, the product is not a desirable coating material. However, prior art processes are difficult to control and prevent from proceeding too far beyond the Amadori rearrangement. Thus, there is need for a simple, reliable and controllable Maillard process to manufacture encapsulated PUFA, lipid-soluble vitamins and lipid-soluble medicaments with improved storage life and stability.

### SUMMARY OF THE INVENTION

This need is met by the present invention. As known in the art, when certain foods are heat treated under moist conditions, Maillard-type reactions can occur. These reactions initially involve a condensation between the carbonyl group of a reducing sugar with the free amino group of an amino acid, protein, urea, fatty amines or other suitable nitrogen source, such as the phosphatidylcholine and phosphatidylethanolamide that are present in phospholipid sources such as lecithin, which is extracted from soybean oil refining. The result is a Maillard reaction product.

The present invention incorporates the discovery that the Maillard reaction, specifically a Maillard reaction conducted at reduced pressure (below atmospheric pressure) and under specific conditions, can be advantageously employed to create stable coated micelles that can be used to enhance shelf life of polyunsaturated fatty acids, lipid-soluble vitamins and/or lipid-soluble medicaments. The present invention is directed to the preparation of coated micelle products that prevent the oxidation of PUFA, such as docosahexaenoic acid (DHA), lipid-soluble vitamins, such as vitamin A, and/or lipid-soluble medicaments.

The coated micelles are generated by using a reduced pressure Maillard reaction between the amino groups of amine-containing phospholipids, such as those found in sources such as lecithin (phosphatidylcholine and phosphatidylethanolamide), where the lecithin has been previously de-oiled by solvent extraction pretreatment, reacting with a reducing sugar normally present in carbohydrates, so that the reaction product coats and protects the PUFA, vitamins or medicaments from exposure to oxygen. The coating thus increases the PUFA shelf life by preventing oxidation and thereby preserving the structural integrity of the fat molecules. The shelf lives of lipid-soluble vitamins and lipid-soluble medicaments are likewise increased.

Therefore, according to one aspect of the invention, a method is provided for preparing a micelle-encapsulated lipid-soluble material that prevents oxidation of the material, the method comprising the steps of:
providing a source of lipid-soluble material that comprises a polyunsaturated fatty acid (PUFA), a lipid-soluble vitamin, a lipid-soluble medicament, or a mixture of two or more thereof;
mixing the lipid-soluble material source with a source of a lipid emulsifying agent having at least one amino group and an emulsifying agent purity of at least 95 wt %, to provide a mixture of the lipid-soluble material, encapsulated by said emulsifying agent;
heating the mixture for a sufficient amount of time at a sufficient temperature and under reduced pressure below 1 atm with sufficient moisture so that a Maillard reaction occurs between the lipid amino groups and the reducing carbohydrate source sufficient to provide a Maillard reaction product coating on the micelles that is effective to prevent oxidation of the lipid-soluble material; and stopping the reaction before it proceeds significantly beyond the Amadori rearrangement steps of the Maillard reaction.

According to another aspect of the invention, a method is provided for preparing an oxidation-resistant micelle-encapsulated PUFA composition with extended shelf life, including the steps of:
mixing a quantity of PUFA with a quantity of an amine-containing lipid source with a purity of at least 95 wt % and a quantity of a reducing carbohydrate source to provide a mixture comprising said reducing carbohydrate source admixed with micelles comprising said PUFA and said amine-containing lipids, wherein the amount by weight of the reducing carbohydrate source is less than either the amount by weight of the PUFA or the amount by weight of the amine-containing lipids, or both;
heating the mixture for a sufficient amount of time at a sufficient temperature and under reduced pressure below 1 atm with sufficient moisture so that a Maillard reaction occurs between the amine-containing lipids and the reducing carbohydrate source sufficient to provide a Maillard reaction product coating on the micelles that is effective to prevent oxidation of the PUFA; and
stopping the reaction before it proceeds significantly beyond the Amadori rearrangement steps of the Maillard reaction.

The nitrogen source for the Maillard reaction resides in the amine-containing lipid having a purity of at least 95 wt %. In one embodiment, the amine-containing lipid is an amine-containing phospholipid. In another embodiment, the amine-containing phospholipid source is lecithin, which contains phosphatidylcholine and phosphatidylethanolamide. Any other N group in lecithin also participates in the reaction. Phospholipid sources include the neat amine-containing phospholipid. Lecithin and other bulk sources of amine-containing phospholipids should be de-oiled and purified to a purity of at least 95 wt % by pretreatment to remove the oil, preferably using solvent extraction.

The present invention includes methods according to which the product of the present invention is made, as well as products made by the inventive method.

Therefore, according to another aspect of the present invention, an emulsified PUFA composition is provided, in which micelles of PUFA are encapsulated by a lipid emulsifying agent having at least one amino group, wherein the micelles are coated with the Maillard reaction product of the amino groups and a reducing sugar.

In one embodiment, of this aspect, the lipid emulsifying agent is an amine-containing phospholipid. In another embodiment, the lipid emulsifying agent is lecithin. According to another embodiment, the amine-containing phospholipids have been de-oiled by solvent extraction.

In one embodiment of this aspect, the PUFA contain DHA. In another embodiment, the PUFA contain EPA. In yet another embodiment, the reducing sugar is selected from fructose, sucrose, dextrose, high fructose corn syrup, glucose, lactose, molasses, xylose, spent sulfite liquor, and mixtures of two or more thereof.

For the Maillard reaction to work efficiently, the lecithin or other phospholipid source must be purified by the pretreatment to remove residual oil, preferably by solvent extraction and centrifuging.

Any other amine-containing lipid emulsifier can be used to create the micelle as long as the emulsifier has an amino group and a purity of at least 95 wt %, which in presence of the carbonyl group from the reducing sugar act as a precursor of the Maillard reaction.

According to one embodiment, PUFA are selected from C18, C20, or C22 fatty acids or derivatives thereof, such as DHA (22:6, n−3) and EPA (20:5, n−3). Derivatives of the fatty acids include esters and a mixture of unsaturated fatty acids and saturated fatty acids.

According to one embodiment, the reducing sugar source is selected from fructose, sucrose, dextrose, high fructose corn syrup, glucose, lactose, molasses, xylose, and spent sulfite liquor.

According to one embodiment, the mixture is heated to a temperature between about 60° C. and about 135° C., or between about 60° C. and about 120° C., or between about 60° C. and about 80° C. The pressure during heating can be between about 0.4 Atm and about 0.9 Atm, or between about 0.4 Atm and about 0.6 Atm, or between about 0.4 Atm and about 0.5 Atm.

The mixture heating time can be about 0.5 min to about 240 min depending on the temperature and pressure used. In one embodiment, the reaction time is between about fifteen seconds and about two hours. In another embodiment, the mixture heating time is about 45 minutes.

The ratio of reducing sugar to lecithin is selected from one of the following weight ratios: about 1:99, about 10:90, about 20:80, about 30:70, about 40:60, about 45:55, or about 50:50. The final product embodiments according to the invention contains one of the following quantities of polyunsaturated fatty acids: greater than about 10 wt %, greater than about 20 wt %, greater than about 30 wt %, greater than about 40 wt %, greater than about 50 wt % greater than about 60 wt %, greater than about 70 wt % or greater than about 80 wt % of the polyunsaturated fatty acid. Each of the foregoing embodiments include embodiments that contain one of the following quantities of polyunsaturated fatty acids: less than about 90 wt %, less than about 80 wt %, less than about 70 wt %, less than about 60 wt % or less than about 50 wt % of polyunsaturated fatty acid.

The method of preparing the micelle can comprise a reactor or an extruder used like a biochemical reactor to accelerate the Maillard reaction before the vacuum cooking and drying. A specialized extruder could be used to produce the mixture, cooking and low-pressure cooking in an extruder with a degassing section where the pressure can be maintained below atmospheric pressure.

The method of preparing the micelle can comprise using a reactor or an extruder as a biochemical reactor to accelerate the Maillard reaction prior to vacuum cooking and drying. The extruder may be used to produce the mixture, including cooking the mixture and cooking the mixture at low pressure below 1 atm in extruder section having a degassing barrel.

According to another embodiment, the method of preparing the PUFA micelles to enhance PUFA shelf life includes the steps of:
mixing de-oiled lecithin and the PUFA with water at a temperature of 25° C. until a uniform, homogenous micelle mixture is formed;
adding the reducing sugar in a quantity less than that of either the de-oiled lecithin or the PUFA, o or both;
continuing mixing until the mixture is homogeneous again; and
heating the mixture for about 15 seconds to about 240 min, at a temperature between about 30° C. and about 145° C. and a pressure between about 0.4 Atm and about 0.9 Atm in the presence of sufficient moisture so that a Maillard reaction product micelle coating is formed in an amount sufficient to coat the micelles with a Maillard reaction product layer that is effective to prevent the PUFA from oxidizing, thereby enhancing its shelf life.

In order to get a stable micelle, PUFA should be prepared by the above-described method. The quantity of reducing sugar used in the reaction mixture can range from about 1% to about 30%, or about 2% to about 25%, or about 3% to about 20% based on the total weight of the mixture.

For easier handling, the micelle can be a liquid product dried onto a matrix. The matrix can be selected from soybean meal, corn meal, silicates, rice hulls, mill run, ground corn, dried corn gluten feed, citrus pulp, oats hulls, sorghum grain, wheat mill run, sunflower meal, wet distillers grains, aluminum silicates, diatomaceous earths, maltodextrins, maltodextrose, wheat midds and mixtures of two or more thereof.

In addition to PUFA and their derivatives, the coated micelle can also encapsulate other fats, fat-soluble vitamins and/or other lipophilic nutrient compounds. As such, the product can be further incorporated into a dietary supplement such as a nutraceutical product for humans. The product can be incorporated, with or without additional vitamin supplements, into a tablet, gel tablet, capsule, or other acceptable mode of administration using methods known to one of ordinary skill. Lipid-soluble medicaments can also likewise be encapsulated and protected from oxidation.

The foregoing and other aspects of the present invention are better appreciated by reference to the detailed description and drawings set forth below.

### BRIEF DESCRIPTION OF THE FIGURES

FIG. 1 shows a schematic structure drawing of a typical PUFA micelle prepared according to the disclosed process.

FIG. 2 shows the Maillard process used to produce the encapsulation.

### DETAILED DESCRIPTION OF THE INVENTION

The term “polyunsaturated fatty acid” or “PUFA” denotes a fatty acid that contains more than one double bond in the backbone. These PUFA generally contain about 16 to about 24 carbon atoms and 2 to 6 double bonds. Representative compounds include Hexadecatrienoic acid (HTA), Alpha-linolenic acid (ALA), Stearidonic acid (SDA), Eicosatrienoic acid (ETE), Eicosatetraenoic acid (ETA), EPA (Timnodonic acid), Heneicosapentaenoic acid (HPA), Docosapentaenoic acid (DPA, Clupanodonic acid), DHA (Cervonic acid), Tetracosapentaenoic acid, Tetracosahexaenoic acid (Nisinic acid), Linoleic acid (LA), Gamma-linolenic acid (GLA), Eicosadienoic acid, Dihomo-gamma-linolenic acid (DGLA), Arachidonic acid (AA), Docosadienoic acid, Adrenic acid (AdA), Docosapentaenoic acid (Osbond acid), Tetracosatetraenoic acid, Tetracosapentaenoic acid, Mead acid, Rumenic acid, α- and β-Calendic acid, Jacaric acid, α- and β-Eleostearic acid, Catalpic acid, Punicic acid, Rumelenic acid, α- and β-Parinaric acid, Bosseopentaenoic acid, Pinolenic acid, and Sciadonic acid.

The term “lipid-soluble vitamin” or “fat-soluble vitamin” denotes a vitamin that can dissolve in fats and oils. Fat-soluble vitamins are absorbed along with fats in the diet and are stored in the body's fatty tissues as well as in the liver. Representative compounds include vitamin A sources, such as all-trans-Retinol, Retinals, and alternative provitamin A-functioning Carotenoids including all-trans-beta-carotene; vitamin D sources, such as Cholecalciferol (D3) and Ergocalciferol (D2); vitamin E sources, such as Tocopherols and Tocotrienols; and vitamin K sources, such as Phylloquinone and Menaquinones.

The term “lipid-soluble medicament” includes “lipid-soluble nutraceuticals” and “lipid-soluble pharmaceuticals”, where the lipid-soluble compounds have a log P (logarithm of the partition coefficient between n-octanol and water; also known as log K_{OW}) of 1 or higher, preferably 2 or higher, and particularly 3 or higher. Lipinski's Rule of 5, an empirical drug discovery rule developed at Pfizer in the 1990s, states that the log P of a compound intended for oral administration should be less than 5.

Lipid-soluble nutraceuticals include, but are not limited to, essential oils, such as basil oil, carrot seed oil, celery seed oil, citronella oil, coriander oil, garlic oil, grapefruit oil, lemon oil, marjoram oil, onion oil, organum oil, parsley oil, rosemary oil, sage oil, tarragon oil, thyme oil, cannabis oil, and any other lipid-soluble plant extract. Two or more of these can also be combined.

Lipid-soluble pharmaceuticals include, without limitation, antibiotics in the tetracycline, sulfonamide, fluoroquinolone, macrolide and licosamine families.

Examples of tetracyclines include, without limitation, chlortetracycline, oxytetracycline, demeclocycline, lymecycline, methacycline, minocycline, rolitetracycline, doxycycline, tigecycline, eravacycline, sarecycline, omadacycline and combinations of two or more.

Examples of sulfonamides include, without limitation, sulfafurazole, Sulfacetamide, Sulfadiazine, Sulfadimidine, Sulfafurazole (sulfisoxazole), Sulfisomidine (sulfaisodimidine), Sulfamethoxazole, Sulfamoxole, Sulfanitran, Sulfadimethoxine, Sulfamethoxypyridazine, Sulfametoxydiazine, Sulfadoxine, Sulfametopyrazine, Terephtyl, Acetohexamide, Carbutamide, Chlorpropamide, Glibenclamide (glyburide), Glibornuride, Gliclazide, Glyclopyramide, Glimepiride, Glipizide, Gliquidone, Glisoxepide, Tolazamide, Tolbutamide, Acetazolamide, Bumetanide, Chlorthalidone, Clopamide, Furosemide, Hydrochlorothiazide, Indapamide, Mefruside, Metolazone, Xipamide, Methazolamide, Ethoxzolamide, Sultiame, Zonisamide, Mafenide, Amprenavir, Darunavir, Delavirdine, Fosamprenavir, Tipranavir, Asunaprevir, Beclabuvir, Dasabuvir, Grazoprevir, Paritaprevir, Simeprevir, Azabon, Apricoxib, Celecoxib, Parecoxib, Bosentan, Dronedarone, Sotalol blocker), Tamsulosin (a blocker), Udenafil (PDE5 inhibitor), Dorzolamide, Probenecid, Sulfasalazine, and Sumatriptan. Two or more of these can also be combined.

Examples of fluoroquinolones include, without limitation, Ciprofloxacin, Garenoxacin, Gatifloxacin, Gemifloxacin, Levofloxacin, and Moxifloxacin. Two or more of these can also be combined.

Examples of macrolides include, without limitation, Azithromycin, Clarithromycin, Erythromycin, Virginiomycine (for ruminants).

Examples of licosamides include, without limitation, Clindamycin, Lincomycin, and Pirlimycin. Two or more of these can also be combined.

For an industrially robust process, it has now been unexpectedly discovered that the preparation of the Maillard reaction product between a 95 wt % purity amine-containing lipid's amino groups and a reducing sugar can be conducted by heating under less than atmospheric pressure (i.e. under vacuum). When the amine-containing lipid, such as an amine-containing phospholipid, is used in the present invention, the phospholipid's oil is removed by a solvent extraction pretreatment.

The Maillard reaction occurs in three stages. In the initial stage, which is reversible, a colorless product, without absorption of ultraviolet light (about 280 nm) is produced through two reactions: sugar-amine condensation and Amadori rearrangement. A product of Amadori rearrangement is 1-amino-1-deoxy-2-ketose, and this product can revert back to a 6 carbon reducing sugar. In an intermediate stage a colorless or yellow product, with strong absorption of ultraviolet light, is produced through three reactions: sugar dehydration, sugar fragmentation, and amino acid degradation (Strecker degradation). During the intermediate stage, 6 carbon sugars are fragmented into 3 carbon sugars, which is irreversible. In the final stage, a highly colored product is formed through two reactions: aldol condensation and aldehyde-amine condensation and formation of heterocyclic nitrogen compounds.

As used in the present invention, the Maillard reaction is stopped before the intermediate stage. That is, the process runs through completion of the Amadori rearrangement but is stopped before significant Strecker degradation. As a result, the majority of the product of the Maillard reaction (e.g. 60% or more) may be 1-amino-1-deoxy-2-ketose. Some of the product (e.g. 20% or less) may be unreacted reducing sugars, and some of the product (e.g. 20% or less) may be overcooked Strecker degradation products. To control the amount of Amadori rearrangement product, the reaction may occur under controlled time, temperature, pH, and pressure. The desired product can be measured by color, smell, and specialized tools including a spectrophotometer and HPLC.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention. The term “about” generally includes up to plus or minus 10% of the indicated number. For example, “about 10%” may indicate a range of 9% to 11%, and “about 20” may mean from 18 to 22. Preferably “about” includes up to plus or minus 6% of the indicated value. Alternatively, “about” includes up to plus or minus 5% of the indicated value. Other meanings of “about” may be apparent from the context, such as rounding off, so, for example “about 1” may also mean from 0.5 to 1.4.

The micelle compositions of the present invention can be in the form of dry fine powders, or liquids. The compositions can be made by weighing and mixing together the component quantities with up to 25% by weight of distilled water, in any equipment suitable for mixing materials. The amine-containing phosphor lipid source are first mixed together to form the micelles, after which the reducing carbohydrate source is added. The mixture is then heated under reduced pressure to between about 60° C. and about 95° C., preferably between about 60° C. and about 90° C., more preferably at about 85° C., at a pressure between about 0.4 and less than 1.0 Atm, preferably at about 0.85 Atm for about 7 min to about 4 hours, preferably between about 30 and about 45 min, and then cooled to room temperature. Table 1, below, indicates the appropriate temperatures and pressures determined to be useful for the reduced pressure Maillard reaction for the formation of a PUFA micelle:

TABLE 1
Reaction conditions for Maillard synthesis of PUFA and lecithin

| Temperature | Pressure |
|---|---|
| (° C.) | (ATM) |
| 100 | 1 |
| 95 | 0.83 |
| 90 | 0.70 |
| 85 | 0.59 |
| 80 | 0.51 |
| 75 | 0.46 |
| 70 | 0.44 |
| 65 | 0.44 |
| 60 | 0.44 |

The quantity of reducing sugar used in the reaction mixture can range from about 1 wt % to about 30 wt %, or about 2 wt % to about 25 wt %, or about 3 wt % to about 20 wt % based on the total weight of the mixture. The quantity of PUFA used in the reaction mixture can range from about 1 wt % to about 80 wt % or about from 10 wt % to about 70 wt %, or from about 20 wt % to about 60 wt % based on the total weight of the mixture. The quantity of amine-containing lipid used in the reaction mixture can range from about 1 wt % to about 80 wt % or about from 10 wt % to about 70 wt %, or from about 20 wt % to about 60 wt % based on the total weight of the mixture. The mixture also includes up to about 25 wt % water and may optionally include carriers, inert ingredients and nutritional supplements for co-delivery with the PUFA. Analogous reactions occur with lipid-soluble vitamins and medicaments.

A typical amine-containing phospholipid source, i.e., lecithin, PUFA and sugar formulation is depicted in Table 2, together with the acceptable ranges within which individual components can be varied:

TABLE 2
Ranges of components in the micelles

| | |
|---|---|
| International | Proportion (g/kg) |

| Ingredient | Feed Number | Preferred¹ | Range |
|---|---|---|---|
| 1. DHA | | 350 | 10-800 |
| 2. Lecithin | | 620 | 300-800 |
| 3. Dextrose | 6-02-633 | 30 | 1-300 |

¹the preferred PUFA is DHA

The micelle compositions of the present invention can also be optionally formulated with alternative sugar sources (other than dextrose) in accordance with availability and pricing of ingredients. Fructose, sucrose, high fructose corn syrup, glucose, lactose, molasses, xylose, and spent sulfite liquor, as well as other reducing sugars can be used as the optional sugar source.

The supplement can also be formulated with a bulk source of PUFA in general, and DHA in particular, such as fish oil, marine algae products, or any other bulk source of PUFA or DHA.

Essentially any amine-containing lipid emulsifier can be used in the present invention, provided that the purity is at least 95 wt %. Neat amine-containing phospholipids can be used, as well as bulk sources of the phospholipids, such as lecithin. For purposes of the present invention, neat amine-containing phospholipids are defined as having a purity of at least 95 wt %. Lecithin and other bulk sources of amine-containing phospholipids should be de-oiled and purified to a purity of at least 95% by pretreatment to remove the oil, preferably using solvent extraction.

Bulk sources of amine-containing phospholipids should be de-oiled by extraction with a solvent capable of separating non-polar oils from more polar compounds, such as acetone, hexane, and related solvents, to remove the residual oil and sterols before mixing with the DHA or other PUFA source. Optionally, the de-oiled amine-containing phospholipid can be further extracted with an alcohol, such as ethanol, to remove residual sugars. In preferred embodiments, the de-oiled lecithin is extracted with ethanol.

The lecithin can be from soybean, sunflower, rape seed, or any other available and well-known lecithin source. Each source has a unique profile of individual phospholipid species as well as fatty acids. Lecithin can be replaced in whole or in part with any other emulsifier that contains amino-groups capable of reacting with the carbonyl groups of the reducing sugar.

When the micelle is prepared in a liquid medium, to produce a liquid product, the resulting liquid product can be applied to and dried onto various matrices, such as soybean meal, corn meal, silicates (verixite, vermiculite, etc.), rice hulls, mill run, ground corn, citrus pulp, oats hulls, sorghum grain, wheat mill run, aluminum silicates, diatomaceous earths, maltodextrins, maltodextrose, dry distillers grains, wet distillers grains, wheat midds, or a blend of two or more of these.

One aspect of the invention is directed to a method of preparing lecithin-emulsified DHA protected from oxidation and rancidity:
mixing de-oiled lecithin with a DHA source, heating and then adding the reducing sugar to the mixture, where the amount by weight of the reducing sugar source versus the amount by weight of the lecithin ranges from about 1:99 to less than 50:50, to provide a mixture; and
heating the mixture for a sufficient amount of time, at a sufficient temperature and under reduced pressure, in the presence of sufficient moisture so that a Maillard reaction product is formed, where the amount of lecithin and the heating time, temperature, reduced pressure and moisture conditions are sufficient to provide an amount of a Maillard reaction product coating effective to prevent hydrogenation of the DHA.

The ratio of reducing sugar to lecithin is selected from one of the following weight ratios: about 1:99, about 10:90, about 20:80, about 30:70, about 40:60, about 45:55, or about 50:50. The final product embodiments according to the invention contain one of the following quantities of polyunsaturated fatty acids: greater than about 10 wt %, greater than about 20 wt %, greater than about 30 wt %, greater than about 40 wt %, greater than about 50 wt % greater than about 60 wt %, greater than about 70 wt % or greater than about 80 wt % of the polyunsaturated fatty acid. Each of the foregoing embodiments include embodiments that contain less than about 90 wt %, less than about 80 wt %, less than about 70 wt %, less than about 60 wt % or less than about 50 wt % of polyunsaturated fatty acid.

The reducing sugar source can be selected from fructose, sucrose, dextrose, high fructose corn syrup, glucose, lactose, molasses, xylose, spent sulfite liquor, any other polysaccharide that can react with an amino group on an emulsifier and mixtures of two or more thereof. The reducing sugar can be present in emulsifiers that use polysaccharides as their reactive agent.

The mixture is heated to a temperature between about 30° C. and about 145° C. Within this range, the mixture can be heated to a range within one of the following temperature ranges: between about 30° C. and about 135° C., between about 30° C. and about 95° C., or between about 60° C. and about 90° C., or between about 60° C. and about 85° C., or between about 60° C. and about 80° C. The mixture can be heated to a temperature between about 40° C. and about 95° C., or about 45° C. and about 90° C., or about 50° C. and about 85° C., or about 55° C. and about 80° C., or about 60° C. and about 75° C. The pressure during heating can be between about 0.4 Atm and about 0.9 Atm. Within this range, the pressure during heating can range between one of the following pressure ranges: about 0.4 Atm and about 0.8 Atm, or between about 0.4 Atm and about 0.9 Atm, or between about 0.4 Atm and about 0.6 Atm, or between about 0.4 Atm and about 0.5 Atm. The pressure during heating can be about 0.4 Atm, or about 0.45 Atm, or about 0.5 Atm, or about 0.55 Atm, or about 0.6 Atm, or about 0.65 Atm, or about 0.7 Atm, or about 0.75 Atm, or about 0.8 Atm, or about 0.85 Atm, or about 0.9 Atm, or about 0.95 Atm.

The mixture heating time can be seconds to minutes to hours. Preferably the mixture heating time is about 7 min to about 120 min. In this embodiment, the mixture heating time can be selected from the following minimum heating times: about 7 min, or about 10 min, or about 15 min, or about 20 min, or about 25 min, or about 30 min, or about 35 min, or about 40 min, or about 45 min, or about 50 min, or about 55 min, or about 60 min, or about 65 min, or about 70 min, or about 75 min, or about 80 min, or about 85 min, or about 90 min. Alternatively, the mixture heating time can be up to about 240 min, with a minimum heating time selected from the following minimum heating times: about 7 min, or about 150 min, or about 175 min, or about 200 min, or about 225 min, or about 240 min.

The method for preparing the coated micelles can comprise a reactor or an extruder used like a reactor to accelerate the Maillard reaction, followed by vacuum cooking and drying. Configuration of an extruder having degassing sections for continuous reaction in the presence of shear mixing is preferred in order to achieve a continuous Maillard reaction process.

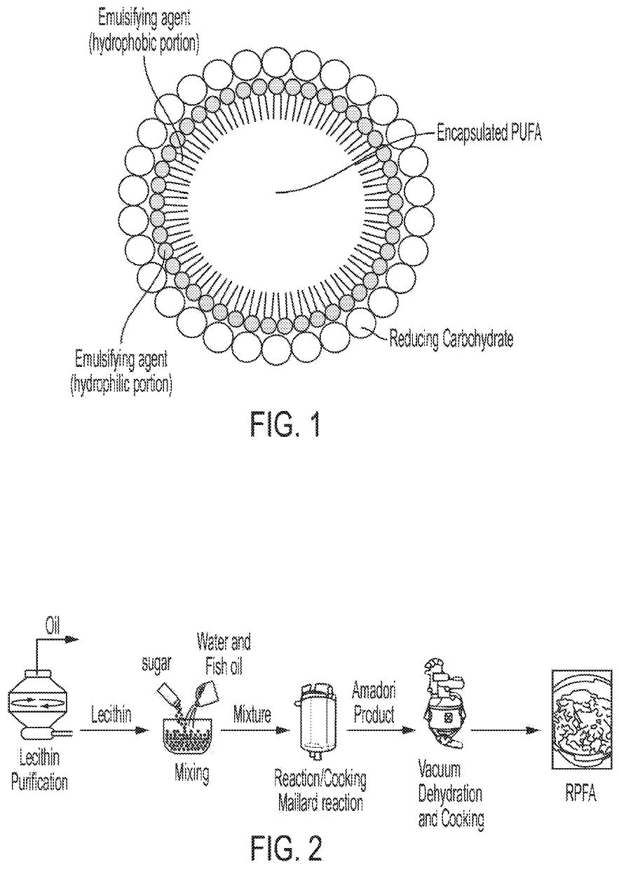

FIG. 1 depicts a micelle formed by the method of the present invention. The inner core of PUFA is surrounded by a layer of an emulsifying agent, for example, the phospholipids in lecithin. The hydrophobic portion of the phospholipids face inward toward the hydrophobic PUFA and the hydrophilic portions of the phospholipids face outwards. The hydrophilic portions of the phospholipids contain amino groups that react with a reducing sugar through a controlled Maillard reaction to form the complete micelle coating.

FIG. 2 shows a schematic of the processing steps used to produce the final product. First the emulsifying agent, such as lecithin, is purified by solvent extraction to remove oils. Next the emulsifier is mixed with a sugar (a reducing sugar) and a source of PUFA, such as fish oil, and water. The Maillard reaction is initiated in a heated vacuum extruder, creating an Amadori product and essentially proceeding no further, which is vacuum cooked and dehydrated, resulting in the final isolated micelle product.

The method of preparing a micelle to enhance shelf life can comprise:
Mixing a reducing sugar source and de-oiled lecithin together with the PUFA to be encapsulated, to provide a mixture; and
heating the mixture for about 7 min to about 120 min (alternatively, up to about 240 min), at a temperature between about 30° C. and about 145° C., and a pressure between about 0.4 Atm and about 0.9 Atm, in the presence of sufficient moisture so that a Maillard reaction product is formed in an amount sufficient to form a Maillard product coated micelle that will prevent oxidation of the PUFA.

The PUFA micelle (or lipid-soluble vitamin, or lipid-soluble medicament micelle) can optionally further comprise a pH adjustment agent. The pH adjustment agent can comprise a buffer. The buffer components can be selected from one or more of sodium bicarbonate, potassium dihydrogen phosphate, dipotassium hydrogen phosphate, sodium hydroxide or phosphoric acid. The buffer can consist essentially of about 50% sodium bicarbonate, about 20% potassium dihydrogen phosphate, and about 30% dipotassium hydrogen phosphate, or 10% sodium hydroxide.

Optionally, the pH of the PUFA, vitamin or medicament composition can be adjusted to about 2 to about 11. Within this range, the pH can range between one of the following pH ranges: about 3 to about 10, about 4 to about 9, about 5 to about 8.5, about 6 to about 8.5, or about 6 to about 8. The pH can be about 2, about 2.5, about 3, about 3.5, about 4, about 4.5, about 5, about 5.5, about 6, about 6.5, about 7, about 7.5, about 8, about 8.5, about 9, about 9.5, or about 10, about 10.5, or about 11. The amount and composition of buffer required to achieve any of the foregoing pH ranges is readily apparent to one of ordinary skill in this art.

Products according to the present invention also include nutritional supplements for humans and other mammals, such as fish oil supplements optionally containing additional fat-soluble vitamins and other fat-soluble nutritional products. Micelle compositions of the present invention can be administered in oral dosage forms such as tablets, capsules (each of which includes sustained release or timed-release formulations), pills, powders, micronized compositions, granules, elixirs, tinctures, suspensions, syrups and emulsions. Encapsulated ingredient(s) are formulated into pharmaceutically acceptable dosage forms by conventional methods known to those of skill in the art.

Methods of preparing oral dosage forms include the step of bringing the coated micelles into association with a carrier and, optionally, one or more accessory ingredients. In general, the formulations are prepared by uniformly and intimately bringing the coated micelles into association with liquid carriers, or finely divided solid carriers, or both, and then, if necessary, shaping the product.

Formulations of the invention suitable for oral administration may be in the form of capsules, cachets, pills, tablets, lozenges (using a flavored base, usually sucrose and acacia or tragacanth), powders, granules, or as a solution or a suspension in an aqueous or non-aqueous liquid, or as an elixir or syrup, or as pastilles (using an inert base, such as gelatin and glycerin, or sucrose and acacia) and the like, each containing a predetermined amount of the coated micelles.

In solid dosage forms of the invention for oral administration (capsules, tablets, pills, dragees, powders, granules and the like), the coated micelles are mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethyl-cellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such as talc, calcium stearate, magnesium stearate, solid polyethylene glycols, sodium lauryl sulfate, and mixtures thereof; and (10) coloring agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered active ingredient(s) moistened with an inert liquid diluent.

Thus, one aspect of the invention is directed to a method for preparing a micelle-encapsulated lipid-soluble material that prevents oxidation of the material, the method comprising the steps of:
providing a source of lipid-soluble material that comprises a PUFA, a lipid-soluble vitamin, a lipid-soluble medicament, or a mixture of two or more thereof;
mixing the lipid-soluble material source with a source of a lipid emulsifying agent having at least one amino group and an emulsifying agent purity of at least 95 wt %, to provide a mixture of the lipid-soluble material, encapsulated by said emulsifying agent;
heating the mixture for a sufficient amount of time at a sufficient temperature and under reduced pressure below 1 atm with sufficient moisture so that a Maillard reaction occurs between the lipid amino groups and the reducing carbohydrate source sufficient to provide a Maillard reaction product coating on the micelles that is effective to prevent oxidation of the lipid-soluble material; and
stopping the reaction before it proceeds significantly beyond the Amadori rearrangement steps of the Maillard reaction.

Another aspect of the invention is directed to a method of preparing a micelle-encapsulated PUFA that prevents oxidation of the PUFA, where the method comprises: mixing a reducing sugar source, a PUFA source, and a source for a lipid emulsifying agent having at least one amino group and an emulsifying agent purity of at least 95 wt %, to provide a mixture of the reducing sugar and micelles comprising the PUFA encapsulated by the emulsifying agent; heating this mixture for a sufficient amount of time at a sufficient temperature and under reduced pressure below 1 atm with sufficient moisture so that a Maillard reaction occurs between the lipid amino groups and the reducing carbohydrate source sufficient to provide a Maillard reaction product coating on the micelles that is effective to prevent oxidation of the PUFA; and stopping the reaction before it proceeds significantly beyond the Amadori rearrangement steps of the Maillard reaction. The amount by weight of the reducing sugar source can be less than the amount by weight of the emulsifying agent source, as well as less than the weight of the PUFA source. The lipid emulsifying agent source can comprise amine-containing phospholipids. The lipid emulsifying agent source can be lecithin. The lecithin can be present in about 50 to 65%, preferably about 55% by weight.

The amine-containing phospholipids of the method can be de-oiled by solvent extraction. The de-oiled emulsifying agent can be further extracted with an alcohol to remove residual sugars; preferably the extraction alcohol is ethanol.

The heating step of the method can comprise: heating the mixture for about 15 seconds to about 240 min, at a temperature between about 30° C. and about 145° C., and pressure between about 0.4 Atm and about 0.9 Atm, in the presence of sufficient moisture so that the Maillard reaction product is formed on the micelles in an amount sufficient to prevent oxidation of the PUFA. The PUFA source can comprise DHA. The PUFA source can comprise EPA.

The reducing sugar source of the method can be selected from the group consisting of fructose, sucrose, dextrose, high fructose corn syrup, glucose, lactose, molasses, xylose, spent sulfite liquor, and mixtures of two or more thereof. In the method, the ratio of reducing sugar to emulsifying agent can be about 10:90, or about 20:80, or about 30:70.

In the method, the mixture can be heated to a temperature between about 60° C. and about 145° C. at a pressure between about 0.4 Atm and about 0.6 Atm. Alternatively, the mixture can be heated to a temperature between about 60° C. and about 145° C. at a pressure between about 0.4 Atm and about 0.5 Atm. The heating time can be about 45 min.

In the method, the micelle coating is sufficient to enhance or lengthen the shelf life of the PUFA.

The method can be performed in an extruder with degassing sections that provide vacuum, which causes the Maillard reaction to proceed more controllably, and the product can be subsequently processed by vacuum cooking and drying.

Another aspect of the invention is directed to an emulsified polyunsaturated fatty acid (PUFA) composition, comprising micelles of PUFA encapsulated by a lipid emulsifying agent having at least one amino group, where the micelles are coated with the Maillard reaction product of the amino groups and a reducing sugar, where the lipid emulsifying agent can comprise amine-containing phospholipids; the lipid emulsifying agent is preferably lecithin, which is preferably present in about 50 to 65%, preferably about 55% by weight. Preferably the amine-containing phospholipids have been de-oiled by solvent extraction.

The PUFA of the composition can comprise DHA and/or EPA.

The reducing sugar used to produce the composition can be selected from the group consisting of fructose, sucrose, dextrose, high fructose corn syrup, glucose, lactose, molasses, xylose, spent sulfite liquor, and mixtures of two or more thereof.

The PUFA composition can be formed into a tablet, gel tablet or capsule.

Another aspect of the invention is directed to a method of preparing a micelle-encapsulated lipid-soluble vitamin that prevents oxidation of the vitamin, where the method comprises: mixing a reducing sugar source, a lipid-soluble vitamin source, and a source for a lipid emulsifying agent having at least one amino group and having an emulsifying agent purity of at least 95 wt %, to provide a mixture of the reducing sugar and micelles comprising the vitamin encapsulated by the emulsifying agent; heating the mixture for a sufficient time at a sufficient temperature and under reduced pressure below 1 atm with sufficient moisture so that a Maillard reaction occurs between the lipid amino groups and the reducing carbohydrate source sufficient to provide a Maillard reaction product coating on the micelles that is effective to prevent oxidation of the vitamin; and stopping the reaction before it proceeds significantly beyond the Amadori rearrangement steps of the Maillard reaction.

Preferably the amount by weight of the reducing sugar source in the method is less than the amount by weight of the emulsifying agent source and is less than the weight of the lipid-soluble vitamin source. The lipid emulsifying agent source preferably comprises amine-containing phospholipids; preferably the emulsifying agent source is lecithin, which can be present in about 50 to 65%, preferably about 55% by weight. Preferably the amine-containing phospholipids have been de-oiled by solvent extraction. Preferably the de-oiled emulsifying agent has been further extracted with an alcohol to remove residual sugars, where the extraction alcohol is preferably ethanol.

Preferably the heating step comprises: heating the mixture for about 15 seconds to about 240 min, at a temperature between about 30° C. and about 145° C., and pressure between about 0.4 Atm and about 0.9 Atm, in the presence of sufficient moisture so that the Maillard reaction product is formed on the micelles in an amount sufficient to prevent oxidation of the lipid-soluble vitamin.

The lipid-soluble vitamin source can comprise a vitamin A source, a vitamin D source, a vitamin E source, a vitamin K source, or a mixture of two or more thereof.

The reducing sugar source can be selected from the group consisting of fructose, sucrose, dextrose, high fructose corn syrup, glucose, lactose, molasses, xylose, spent sulfite liquor, and mixtures of two or more thereof.

Preferably the mixture is heated to a temperature between about 60° C. and about 145° C. at a pressure between about 0.4 Atm and about 0.6 Atm, or at a pressure between about 0.4 Atm and about 0.5 Atm. The heating time can be about 45 min.

In the method, the ratio of reducing sugar to emulsifying agent can be about 10:90, or about 20:80, or about 30:70.

The method provides a micelle coating sufficient to enhance or lengthen the shelf life of the lipid-soluble vitamin.

Preferably the Maillard reaction is performed in an extruder with one or more degassing sections, and the product is subsequently processed by vacuum cooking, and drying.

A further aspect of the invention is directed to an emulsified lipid-soluble vitamin composition, comprising micelles of lipid-soluble vitamin encapsulated by a lipid emulsifying agent having at least one amino group, where the micelles are coated with the Maillard reaction product of the amino groups and a reducing sugar. Preferably the lipid emulsifying agent comprises amine-containing phospholipids; preferably the lipid emulsifying agent is lecithin, which can be present in about 50 to 65%, preferably about 55% by weight. Preferably the amine-containing phospholipids have been de-oiled by solvent extraction.

The composition comprises a lipid-soluble vitamin A source, a vitamin D source, a vitamin E source, a vitamin K source, or a mixture of two or more thereof.

Preferably the reducing sugar used in the Maillard process is selected from the group consisting of fructose, sucrose, dextrose, high fructose corn syrup, glucose, lactose, molasses, xylose, spent sulfite liquor, and mixtures of two or more thereof.

The lipid-soluble vitamin composition can be formed into tablets, gel tablets or capsules.

Yet another aspect of the invention is directed to a method of preparing a micelle-encapsulated PUFA and lipid-soluble vitamin mixture that prevents oxidation of both the PUFA and vitamin, where the method comprises: mixing a reducing sugar source, a PUFA source, a lipid-soluble vitamin source, and a source for a lipid emulsifying agent having at least one amino group and an emulsifying agent purity of at least 95 wt %, to provide a mixture of the reducing sugar and micelles comprising the PUFA and vitamin encapsulated by the emulsifying agent; heating the mixture for a sufficient time at a sufficient temperature and under reduced pressure below 1 atm with sufficient moisture so that a Maillard reaction occurs between the lipid amino groups and the reducing carbohydrate source sufficient to provide a Maillard reaction product coating on the micelles that is effective to prevent oxidation of the PUFA and vitamin; and stopping the reaction before it proceeds significantly beyond the Amadori rearrangement steps of the Maillard reaction.

Preferably the amount by weight of the reducing sugar source is less than the amount by weight of the emulsifying agent source and is less than the combined weight of the PUFA and lipid-soluble vitamin sources.

Preferably the lipid emulsifying agent source comprises amine-containing phospholipids. Preferably the lipid emulsifying agent source is lecithin, which can be present in about 50 to 65%, preferably about 55% by weight. Preferably the amine-containing phospholipids have been de-oiled by solvent extraction. Preferably the de-oiled emulsifying agent has been further extracted with an alcohol to remove residual sugars; the extractant alcohol is preferably ethanol.

The heating step of the method can comprise: heating the mixture for about 15 seconds to about 240 min, at a temperature between about 30° C. and about 145° C., and pressure between about 0.4 Atm and about 0.9 Atm, in the presence of sufficient moisture so that the Maillard reaction product is formed on the micelles in an amount sufficient to prevent oxidation of the PUFA and vitamin.

Preferably the PUFA source comprises DHA and/or EPA. Preferably the lipid-soluble vitamin source comprises a vitamin A source, a vitamin D source, a vitamin E source, a vitamin K source, or a mixture of two or more thereof.

Preferably the reducing sugar source is selected from the group consisting of fructose, sucrose, dextrose, high fructose corn syrup, glucose, lactose, molasses, xylose, spent sulfite liquor, and mixtures of two or more thereof.

Preferably the mixture is heated to a temperature between about 60° C. and about 145° C. at a pressure between about 0.4 Atm and about 0.6 Atm, or about 0.4 Atm and about 0.5 Atm. Preferably the heating time is about 45 min.

Preferably the ratio of reducing sugar to emulsifying agent is about 10:90, or about 20:80, or about 30:70.

The method provides a micelle coating that is sufficient to enhance or lengthen the shelf life of both the PUFA and the lipid-soluble vitamin.

Preferably the Maillard reaction is performed in an extruder having one or more degassing sections, where the product is subsequently processed by vacuum cooking, and drying.

Still another aspect of the invention is directed to an emulsified PUFA/lipid-soluble vitamin mixture composition, comprising micelles of a mixture of PUFA and lipid-soluble vitamin encapsulated by a lipid emulsifying agent having at least one amino group, where the micelles are coated with the Maillard reaction product of the amino groups and a reducing sugar.

Preferably the lipid emulsifying agent comprises amine-containing phospholipids. Preferably the lipid emulsifying agent is lecithin, which can be present in about 50 to 65%, preferably about 55% by weight. Preferably the amine-containing phospholipids have been de-oiled by solvent extraction.

Preferably the PUFA of the mixture composition comprises DHA and/or EPA.

Preferably the lipid-soluble vitamin source of the mixture composition comprises a vitamin A source, a vitamin D source, a vitamin E source, a vitamin K source, or a mixture of two or more thereof.

The reducing sugar used to prepare the mixture composition is selected from the group consisting of fructose, sucrose, dextrose, high fructose corn syrup, glucose, lactose, molasses, xylose, spent sulfite liquor, and mixtures of two or more thereof.

The PUFA/lipid-soluble vitamin mixture composition can be formed into tablets, gel tablets or capsules.

A further aspect of the invention is directed to a method of preparing a micelle-encapsulated lipid-soluble medicament that prevents oxidation of the medicament, the method comprising:
mixing a reducing sugar source, a lipid-soluble medicament source, and a source for a lipid emulsifying agent having at least one amino group and an emulsifying agent purity of at least 95 wt %, to provide a mixture of the reducing sugar and micelles comprising said medicament encapsulated by the emulsifying agent;
heating the mixture for a sufficient amount of time at a sufficient temperature and under reduced pressure below 1 atm with sufficient moisture so that a Maillard reaction occurs between the lipid amino groups and the reducing carbohydrate source sufficient to provide a Maillard reaction product coating on the micelles that is effective to prevent oxidation of the medicament; and
stopping the reaction before it proceeds significantly beyond the Amadori rearrangement steps of the Maillard reaction.

Another aspect of the invention is directed to an emulsified lipid-soluble medicament composition, comprising micelles of lipid-soluble medicament encapsulated by a lipid emulsifying agent having at least one amino group, wherein the micelles are coated with the Maillard reaction product of said amino groups and a reducing sugar.

The compositions of the present invention are further illustrated by the following examples. Unless otherwise specified all starting materials and reagents are of standard commercial grade or are readily prepared from such materials by routine methods. Those skilled in the art will recognize that starting materials and reaction conditions may be varied to achieve the desired end product.

### EXAMPLES

### Example 1. Purification of Lecithin

The typical ranges of soybean lecithin components are as follows:

TABLE 3
Typical ranges of soybean lecithin components

| Component | Weight % |
|---|---|
| Phosphatidylcholine | 19-21 |
| Phosphatidylethanolamine | 8-20 |
| Inositol Phosphatides | 20-21 |
| Other Phosphatides | 5-11 |
| Soybean Oil | 33-35 |
| Sterols | 2-5 |
| Free Sugars | ca. 5 |
| Water | ca. 1 |

Soybean lecithin was purified by de-oiling using solvent extraction, which removed residual soybean oil and sterols. The lecithin was mixed with a suitable solvent, typically acetone or hexane, typically in a weight ratio of about 1:1 lecithin: solvent. After a suitable mixing time, the solid is separated from the liquid phase. The solid lecithin residue is recovered in about 65% yield after drying, based on the original lecithin. The de-oiled lecithin residue can be further purified to remove sugars by extracting with an alcohol, such as ethanol.

Method of lecithin purification: Lecithin obtained from an extrusion and press process was mixed with acetone in a proportion of 100 g of lecithin and 30 g of acetone and mixed for 5 minutes. This suspension was centrifuged for 90 minutes at 300 RPM. The suspension separated into two phases, the supernatant, that is the acetone plus the residual oil, was discarded, and the solid part used in the mixtures prepared for the experiments.

The extraction with acetone yields a substance that is 76% ether extract and has at least 1.2% Nitrogen, 9% sugars and 6% water. The composition of the fatty acids is displayed in Table 4:

| | Fatty acids % in Lecithin |
|---|---|
| 16:0 | 15.23 |
| 18:0 | 4.57 |
| 22:0 | 0.4 |
| c9 18:1 | 14.97 |
| c11 18:1 | 1.1 |
| c9c12 18:2n-6 | 53.86 |
| 18:3 n-3 | 7.73 |

### Example 2. Typical Preparation of PUFA Micelle

The purified lecithin of Example 1 is mixed according to Table 5 with a PUFA source, typically fish oil or algae, typically in a weight ratio of about 65:35 purified lecithin:PUFA source. The mixture is heated to about 30° C. and the reducing sugar is added together with enough water to give a moisture content of about 20% to 25% by weight. Heating is continued at about 85° C. for about 2 hours under a vacuum of 0.85 Atm until the water has evaporated, providing the coated PUFA.

TABLE 5
Typical ranges of components for coated DHA

| | International | Proportion (g/kg) | |
|---|---|---|---|
| Ingredient | Feed Number | Preferred¹ | Range |
| 1. Fish Oil (20% DHA) | | 350 | 10-800 |
| 2. Lecithin | | 550 | 300-800 |
| 3. Dextrose | 6-02-633 | 100 | 1-300 |

The coated PUFA, e.g. coated DHA is optionally mixed with a carrier, such as bentonite.

### Example 3. Determination of how the Methodology to Coat PUFA Affects Fatty Acid Composition of the Final Product

Materials and Methods

Lecithin Purification.

Lecithin obtained from extrusion and screw press processes was mixed with acetone in a proportion of 100 g of lecithin and 30 g of acetone and mixed for 5 minutes. This suspension was centrifuged for 90 minutes at 300 RPM. The suspension separated into two phases, the supernatant, that is the acetone plus the residual oil, was discarded, and the solid part, lecithin, was used in the mixtures prepared for the following experiments.

Coating Preparation.

Twenty grams of distilled water was heated to reach 60° C., then 4 g of dextrose was added and mixed for 5 minutes. This solution was added to a mixture of 32 g of flaxseed oil, 44 g of purified soybean lecithin and mixed in a blender during 20 minutes at room temperature (25° C.).

The mixture was then heated under vacuum at 85° C. and 0.8 atm until the dry mater of the mix was 95%, approximately 2 hs in time.

Gas Chromatography Analysis.

The fatty acid composition of samples was determined using 100 to 150 mg of both cooked and uncooked product using a previous method described by O'Fallon et al. (2007, J. Anim. Sci. 85:1511-1521.) with slight modifications (Coleman et al., 2018; J. Anim. Sci. 96:1181-1204).

In a glass Pyrex tube with screw caps, 0.5 g of ground sample, 1 mL of internal standard (C19:0) at 0.5 mg/mL (Nu-Chek Prep, Inc. Elysian, MN), 0.7 mL of 10N KOH in water, and 5.3 mL ethanol were vortexed for 120 s. The samples were heated at 55° C. in a water bath for 1.5 hr with rigorous shaking for 5 s every 20 min. Samples were removed from the bath and transferred to an ice water bath to cool below room temperature. After cooling, 0.58 mL of 24 N H₂SO₄ was added to each sample. Samples were then mixed by inversion and incubated for 1.5 hr in a 55° C. water bath with rigorous shaking for 5 s every 20 min.

Samples were transferred into a cool water bath to cool below room temperature. Then, 3 mL of hexane was added to the samples and the tubes were vortexed for 5 min. The hexane layer was placed in a GC vial and stored at −20° C. until GC analysis. All fatty acid methyl esters were separated by gas-liquid chromatography using a CP-SIL88 capillary column (100-m×0.25-mm×0.2-μm film thickness; Varian Inc., Palo Alto, CA).

Results.

As it is shown in table 6 the PUFA contents in the uncooked molecule are almost exactly the same as the cooked molecule.

TABLE 6
Fatty acid composition of cooked and uncooked product

| | Uncooked Product | Cooked product |
|---|---|---|
| 14:0 | 0.08 | 0.08 |
| 16:0 | 10.63 | 10.47 |
| C9 16:1 | 0.1 | 0.09 |
| 17:0 | 0.1 | 0.1 |
| 18:0 | 5.05 | 5.02 |
| 20:0 | 0..26 | 0.26 |
| 22:0 | 0.34 | 0.34 |
| 24:0 | 0.18 | 0.18 |
| c9 18:1 | 18.39 | 17.91 |
| c11 18:1 | 0.93 | 1.24 |
| c9c12 18:2n-6 | 36.57 | 35.73 |
| 18:3 n-3 | 27.43 | 28.21 |

Conclusion: The method of preparing the coated PUFA does not affect fatty acid profile in the final product.

### Example 4. Determination of the Oxidative Stability of Coated Flaxseed Oil Stored for 6 Months at Room Temperature

Materials and Methods

Coating Preparation.

Twenty grams of distilled water was heated at 60° C. for 5 minutes then 4 g of dextrose was added and mixed, this solution was added to a mixture of 32 g of flaxseed oil, 44 g of treated soybean lecithin and mixed for 20 minutes.

The mix was then heated under vacuum (0.8 atm) and 85° C. until the dry mater was 95%, this process step took approximately 2 hs of time.

The product was left in a container at shelf temperature for 6 months.

Oxidative Stability Testing. p-Anisidine value.

The total oil was extracted by dissolving 180 mg powder in 1.8 mL double distilled water. A volume of 2 mL iso-octane:2-propanol (3:1 v/v) was added and vortexed. Finally, it was centrifuged at 2000×g for 15 min.

Primary oxidation products in form of hydroperoxides were determined by a spectrophotometric determination of colored ferric complexes. For the secondary oxidation compounds the p-anisidine value (pAV) was measured based on the AOCS Official Methods Cd 18-90 with slight modifications.

Results.

The p-anisidine method is used to measure the oxidation (rancidity) of oils. The p-anisidine value increased from 2-3 to 7-9 since non-encapsulated oils are susceptible to environmental oxygen and oxidation. However, pAV values of 7-9 do not imply high rancidity. In fact, these values are still below the rancid limit, which specifies a pAV of 20.

TABLE 7
p-Anisidine test results for RPFA (encapsulated flaxseed oil)

| Sample | pAV |
|---|---|
| 1 | 8.24 |
| 2 | 9.16 |
| 3 | 7.83 |
| 4 | 8.48 |

Conclusion: The oxidative stability test yielded results that indicate low oxidation of the coated fatty acids.

### Example 5. Determination of Oxidation Stability of Coated Flax Seed Oil Stored During 6 Months at Room Temperature when Surface Excess Oil was Removed from the Coating

Materials and Methods

Coating Preparation.

Twenty grams of distilled water was heated at 60° C. for 5 minutes then 4 g of dextrose was added and mixed until to form a solution. The solution was added to a mixture of 32 g of flaxseed oil, 44 g of treated soybean lecithin and mixed during 20 minutes.

The mix was then heated under vacuum at 85° C. and 0.8 atm until the dry mater of the mix was 95%, approximately the time for this step was about 2 hs in time.

The preparation was left in a container at shelf temperature for 6 months.

Removal of Surface Free Oil.

One gram of the coated product was accurately weighed on a filter paper (No. 4, Whatman, Maidstone, Kent, UK), and washed with 5 mL of petroleum ether (bp 40-60° C.). This step typically took approximately 10 seconds. The residue was dried under vacuum at room temperature, and the filtrated solution containing the extracted oil was allowed to evaporate until the extracted fat residue achieved a constant weight. The extracted oil value was then recorded as grams of surface free-oil/g fresh powder.

Once enough product was obtained the oxidative stability test was repeated. Oxidative Stability. p-Anisidine value.

The total oil was extracted by dissolving 180 mg powder in 1.8 mL double distilled water. A volume of 2 mL iso-octane:2-propanol (3:1 v/v) was added and the mixture vortexed. Finally, it was centrifuged at 2000×g for 15 min.

Primary oxidation products in form of hydroperoxides were determined by a spectrophotometric determination of coloured ferric complexes. For secondary oxidation compounds the p-anisidine value (pAV) was measured based on the AOCS Official Methods Cd 18-90 with slight modifications.

Results.

Table 8 illustrates results from coated PUFA after excess soy oil and flaxseed oil was removed from the coating; pAV values drop to less than 3 showing that the coating protects the flaxseed oil from oxidation.

TABLE 8
p-Anisidine test results after removing surface oil of RPFA

| Washed Sample | pAV |
|---|---|
| 1 | 2.39 |
| 2 | 2.38 |
| 3 | 3.03 |
| 4 | 2.28 |

Conclusion: The coating was able to protect the oil from hydrogenation providing oxidative stability.

### Example 6. Determination of Microbiological Stability of Flaxseed Oil after the Coating Process Stored 6 Months at Room Temperature

Materials and Methods

Coating Preparation.

Twenty grams of distilled water was heated at 60° C. for 5 minutes then 4 g of dextrose was added and mixed this solution was added to a mixture of 32 g of flaxseed oil, 44 g of treated soybean lecithin and mixed for 20 minutes.

The mix was then heated under vacuum at 85° C. and 0.8 atm until the dry mater of the mix was 95% approximately 2 hs in time.

The preparation was stored in a container at room temperature for 6 months.

Microbiological Stability Measurement

Water activity is monitored the in stored samples to determine the microbiological stability of a sample so to assure the food safety of the samples. A portable device (Labswift, Novasina AG, Switzerland) designed quick determination of water activity of food products was utilized.

Results.

TABLE 9
Water activity in coated flaxseed oil

| Sample | Water activity |
|---|---|
| 1 | 0.43 |
| 2 | 0.45 |
| 3 | 0.55 |
| 4 | 0.55 |
| average | 0.495 |

Conclusion: Water activity resulted low showing microbiological stability of the coating process. Coating of the process prevent lipid oxidation that normally occurs at water activities lower than the one found in the coated samples.

While certain of the embodiments of the present invention have been described and specifically exemplified above, it is not intended that the invention be limited to such embodiments. Various modifications may be made thereto without departing from the scope and spirit of the present invention as set forth in the following claims. All such modifications coming within the scope of the present claims are intended to be included herein.

We claim:

1. A method of preparing a micelle-encapsulated lipid-soluble material that prevents oxidation of the material, said method comprising:
providing a source of lipid-soluble material that comprises a polyunsaturated fatty acid (PUFA), a lipid-soluble vitamin, a lipid-soluble medicament, or a mixture of two or more thereof;
mixing the lipid-soluble material source with a source of a lipid emulsifying agent having at least one amino group and an emulsifying agent purity of at least 95 wt %, to provide a mixture of the lipid-soluble material, encapsulated by said emulsifying agent;
heating the mixture for a sufficient amount of time at a sufficient temperature and under reduced pressure below 1 atm with sufficient moisture so that a Maillard reaction occurs between the lipid amino groups and the reducing carbohydrate source sufficient to provide a Maillard reaction product coating on the micelles that is effective to prevent oxidation of the lipid-soluble material; and
stopping the reaction before it proceeds significantly beyond the Amadori rearrangement steps of the Maillard reaction.

2. The method of claim 1, wherein the amount by weight of the reducing sugar source is less than the amount by weight of the emulsifying agent source, and is less than the weight of the lipid-soluble material.

3. The method of claim 1, wherein the lipid emulsifying agent source comprises amine-containing phospholipids.

4. The method of claim 3, wherein the lipid emulsifying agent source is lecithin.

5. The method of claim 4, wherein lecithin is present in about 55% by weight.

6. The method of claim 3, wherein the amine-containing phospholipids have been de-oiled by solvent extraction.

7. The method of claim 6, wherein said de-oiled emulsifying agent has been further extracted with an alcohol to remove residual sugars.

8. The method of claim 7, where said alcohol is ethanol.

9. The method of claim 1, wherein said heating step comprises:
heating the mixture for about 15 seconds to about 240 min, at a temperature between about 30° C. and about 145° C., and pressure between about 0.4 Atm and about 0.9 Atm, in the presence of sufficient moisture so that said Maillard reaction product is formed on said micelles in an amount sufficient to prevent oxidation of said lipid-soluble material.

10. The method of claim 1 or 9, where said lipid-soluble material source comprises a PUFA.

11. The method of claim 10, wherein said PUFA comprises EPA and/or DHA.

12. The method of claim 1 or 9, wherein the reducing sugar source is selected from the group consisting of fructose, sucrose, dextrose, high fructose corn syrup, glucose, lactose, molasses, xylose, spent sulfite liquor, and mixtures of two or more thereof.

13. The method of any one of claims 1 to 12, wherein the mixture is heated to a temperature between about 60° C. and about 145° C. at a pressure between about 0.4 Atm and about 0.6 Atm.

14. The method of claim 13, wherein the mixture is heated to a temperature between about 60° C. and about 145° C. at a pressure between about 0.4 Atm and about 0.5 Atm.

15. The method of claim 14, wherein the heating time is about 45 min.

16. The method of any one of claims 1 to 15, wherein the ratio of reducing sugar to emulsifying agent is about 10:90.

17. The method of any one of claims 1 to 15, wherein the ratio of reducing sugar to emulsifying agent is about 20:80.

18. The method of any one of claims 1 to 15, wherein the ratio of reducing sugar to emulsifying agent is about 30:70.

19. The method of claim 1 or 9, wherein the micelle coating is sufficient to enhance or lengthen the shelf life of the PUFA, lipid-soluble vitamin, lipid-soluble medicament or mixture thereof.

20. The method of claim 1 or 9, wherein the emulsifying agent is lecithin.

21. The method of any one of claim 1 or 9, wherein the Maillard reaction is performed in an extruder having one or more degassing sections.

22. The method of claim 1 or 9, wherein the product is subsequently processed by vacuum cooking and drying.

23. An emulsified PUFA composition, comprising micelles of PUFA encapsulated by a lipid emulsifying agent having at least one amino group, wherein the micelles are coated with the Maillard reaction product of said amino groups and a reducing sugar.

24. The composition of claim 23, wherein the lipid emulsifying agent comprises amine-containing phospholipids.

25. The composition of claim 24, wherein the lipid emulsifying agent is lecithin.

26. The composition of claim 25, wherein lecithin is present in about 55% by weight.

27. The composition of claim 24, wherein the amine-containing phospholipids have been de-oiled by solvent extraction.

28. The composition of claim 23, where said PUFA comprise DHA.

29. The composition of claim 23, wherein said PUFA comprise EPA.

30. The composition of claim 23, wherein the reducing sugar is selected from the group consisting of fructose, sucrose, dextrose, high fructose corn syrup, glucose, lactose, molasses, xylose, spent sulfite liquor, and mixtures of two or more thereof.

31. A tablet, gel tablet or capsule comprising the composition of any one of claims 23 to 30.

32. A method of preparing a micelle-encapsulated lipid-soluble vitamin that prevents oxidation of the vitamin, said method comprising:
mixing a reducing sugar source, a lipid-soluble vitamin source, and a source for a lipid emulsifying agent having at least one amino group and an emulsifying agent purity of at least 95 wt %, to provide a mixture of said reducing sugar and micelles comprising said vitamin encapsulated by said emulsifying agent;
heating the mixture for a sufficient amount of time at a sufficient temperature and under reduced pressure below 1 atm with sufficient moisture so that a Maillard reaction occurs between the lipid amino groups and the reducing carbohydrate source sufficient to provide a Maillard reaction product coating on the micelles that is effective to prevent oxidation of the vitamin; and
stopping the reaction before it proceeds significantly beyond the Amadori rearrangement steps of the Maillard reaction.

33. The method of claim 32, wherein the amount by weight of the reducing sugar source is less than the amount by weight of the emulsifying agent source and is less than the weight of the PUFA source.

34. The method of claim 32, wherein the lipid emulsifying agent source comprises amine-containing phospholipids.

35. The method of claim 34, wherein the lipid emulsifying agent source is lecithin.

36. The method of claim 34, wherein lecithin is present in about 55% by weight.

37. The method of claim 34, wherein the amine-containing phospholipids have been de-oiled by solvent extraction.

38. The method of claim 37, wherein said de-oiled emulsifying agent has been further extracted with an alcohol to remove residual sugars.

39. The method of claim 38, where said alcohol is ethanol.

40. The method of claim 32, wherein said heating step comprises:
heating the mixture for about 15 seconds to about 240 min, at a temperature between about 30° C. and about 145° C., and pressure between about 0.4 Atm and about 0.9 Atm, in the presence of sufficient moisture so that said Maillard reaction product is formed on said micelles in an amount sufficient to prevent oxidation of said PUFA.

41. The method of claim 32 or 40, where said lipid-soluble vitamin source comprises a vitamin A source, a vitamin D source, a vitamin E source, a vitamin K source, or a mixture of two or more thereof.

42. The method of claim 32 or 40, wherein the reducing sugar source is selected from the group consisting of fructose, sucrose, dextrose, high fructose corn syrup, glucose, lactose, molasses, xylose, spent sulfite liquor, and mixtures of two or more thereof.

43. The method of any one of claims 32 to 42, wherein the mixture is heated to a temperature between about 60° C. and about 145° C. at a pressure between about 0.4 Atm and about 0.6 Atm.

44. The method of claim 43, wherein the mixture is heated to a temperature between about 60° C. and about 145° C. at a pressure between about 0.4 Atm and about 0.5 Atm.

45. The method of claim 44, wherein the heating time is about 45 min.

46. The method of any one of claims 32 to 45, wherein the ratio of reducing sugar to emulsifying agent is about 10:90.

47. The method of any one of claims 32 to 45, wherein the ratio of reducing sugar to emulsifying agent is about 20:80.

48. The method of any one of claims 32 to 45, wherein the ratio of reducing sugar to emulsifying agent is about 30:70.

49. The method of claim 32 or 40, wherein the micelle coating is sufficient to enhance or lengthen the shelf life of the vitamin.

50. The method of claim 32 or 40, wherein the emulsifying agent is lecithin.

51. The method of any one of claim 32 or 40, wherein the Maillard reaction is performed in an extruder. having one or more degassing section.

52. The method of claim 32 or 40, wherein the product is subsequently processed by vacuum cooking and drying.

53. An emulsified lipid-soluble vitamin composition, comprising micelles of lipid-soluble vitamin encapsulated by a lipid emulsifying agent having at least one amino group, wherein the micelles are coated with the Maillard reaction product of said amino groups and a reducing sugar.

54. The composition of claim 53, wherein the lipid emulsifying agent comprises amine-containing phospholipids.

55. The composition of claim 54, wherein the lipid emulsifying agent is lecithin.

56. The composition of claim 55, wherein lecithin is present in about 55% by weight.

57. The composition of claim 54, wherein the amine-containing phospholipids have been de-oiled by solvent extraction.

58. The composition of claim 53, where said lipid-soluble vitamin comprises a vitamin A source, a vitamin D source, a vitamin E source, a vitamin K source, or a mixture of two or more thereof.

59. The composition of claim 53, wherein the reducing sugar is selected from the group consisting of fructose, sucrose, dextrose, high fructose corn syrup, glucose, lactose, molasses, xylose, spent sulfite liquor, and mixtures of two or more thereof.

60. A tablet, gel tablet or capsule comprising the composition of any one of claims 53 to 59.

61. A method of preparing a micelle-encapsulated polyunsaturated fatty acid (PUFA) and lipid-soluble vitamin mixture that prevents oxidation of both the PUFA and vitamin, said method comprising:
mixing a reducing sugar source, a PUFA source, a lipid-soluble vitamin source, and a source for a lipid emulsifying agent having at least one amino group and an emulsifying agent purity of at least 95 wt %, to provide a mixture of said reducing sugar and micelles comprising said PUFA and vitamin encapsulated by said emulsifying agent;
heating the mixture for a sufficient amount of time at a sufficient temperature and under reduced pressure below 1 atm with sufficient moisture so that a Maillard reaction occurs between the lipid amino groups and the reducing carbohydrate source sufficient to provide a Maillard reaction product coating on the micelles that is effective to prevent oxidation of the PUFA and vitamin; and
stopping the reaction before it proceeds significantly beyond the Amadori rearrangement steps of the Maillard reaction.

62. The method of claim 61, wherein the amount by weight of the reducing sugar source is less than the amount by weight of the emulsifying agent source and is less than the combined weight of the PUFA and lipid-soluble vitamin sources.

63. The method of claim 61, wherein the lipid emulsifying agent source comprises amine-containing phospholipids.

64. The method of claim 63, wherein the lipid emulsifying agent source is lecithin.

65. The method of claim 64, wherein lecithin is present in about 55% by weight.

66. The method of claim 63, wherein the amine-containing phospholipids have been de-oiled by solvent extraction.

67. The method of claim 66, wherein said de-oiled emulsifying agent has been further extracted with an alcohol to remove residual sugars.

68. The method of claim 67, where said alcohol is ethanol.

69. The method of claim 61, wherein said heating step comprises:
heating the mixture for about 15 seconds to about 240 min, at a temperature between about 30° C. and about 145° C., and pressure between about 0.4 Atm and about 0.9 Atm, in the presence of sufficient moisture so that said Maillard reaction product is formed on said micelles in an amount sufficient to prevent oxidation of said PUFA and vitamin.

70. The method of claim 61 or 69, where said PUFA source comprises DHA.

71. The method of claim 61 or 69, wherein said PUFA source comprises EPA.

72. The method of claim 61 or 69, where said lipid-soluble vitamin source comprises a vitamin A source, a vitamin D source, a vitamin E source, a vitamin K source, or a mixture of two or more thereof.

73. The method of claim 61 or 69, wherein the reducing sugar source is selected from the group consisting of fructose, sucrose, dextrose, high fructose corn syrup, glucose, lactose, molasses, xylose, spent sulfite liquor, and mixtures of two or more thereof.

74. The method of any one of claims 61 to 73, wherein the mixture is heated to a temperature between about 60° C. and about 145° C. at a pressure between about 0.4 Atm and about 0.6 Atm.

75. The method of claim 74, wherein the mixture is heated to a temperature between about 60° C. and about 145° C. at a pressure between about 0.4 Atm and about 0.5 Atm.

76. The method of claim 75, wherein the heating time is about 45 min.

77. The method of any one of claims 61 to 76, wherein the ratio of reducing sugar to emulsifying agent is about 10:90.

78. The method of any one of claims 61 to 76, wherein the ratio of reducing sugar to emulsifying agent is about 20:80.

79. The method of any one of claims 61 to 76, wherein the ratio of reducing sugar to emulsifying agent is about 30:70.

80. The method of claim 61 or 69, wherein the micelle coating is sufficient to enhance or lengthen the shelf life of both the PUFA and the lipid-soluble vitamin.

81. The method of claim 61 or 69, wherein the emulsifying agent is lecithin.

82. The method of any one of claim 61 or 69, wherein the Maillard reaction is performed in an extruder having one or more degassing sections.

83. The method of claim 61 or 69, wherein the product is subsequently processed by vacuum cooking and drying.

84. An emulsified PUFA composition, comprising micelles of PUFA encapsulated by a lipid emulsifying agent having at least one amino group, wherein the micelles are coated with the Maillard reaction product of said amino groups and a reducing sugar.

85. The composition of claim 84, wherein the lipid emulsifying agent comprises amine-containing phospholipids.

86. The composition of claim 85, wherein the lipid emulsifying agent is lecithin.

87. The composition of claim 86, wherein lecithin is present in about 55% by weight.

88. The composition of claim 85, wherein the amine-containing phospholipids have been de-oiled by solvent extraction.

89. The composition of claim 84, where said PUFA comprise DHA.

90. The composition of claim 84, wherein said PUFA comprise EPA.

91. The method of claim 84, where said lipid-soluble vitamin source comprises a vitamin A source, a vitamin D source, a vitamin E source, a vitamin K source, or a mixture of two or more thereof.

92. The composition of claim 84, wherein the reducing sugar is selected from the group consisting of fructose, sucrose, dextrose, high fructose corn syrup, glucose, lactose, molasses, xylose, spent sulfite liquor, and mixtures of two or more thereof.

93. A tablet, gel tablet or capsule comprising the composition of any one of claims 84 to 92.

94. A method of preparing a micelle-encapsulated lipid-soluble medicament that prevents oxidation of the medicament, said method comprising:
mixing a reducing sugar source, a lipid-soluble medicament source, and a source for a lipid emulsifying agent having at least one amino group and an emulsifying agent purity of at least 95 wt %, to provide a mixture of said reducing sugar and micelles comprising said medicament encapsulated by said emulsifying agent;
heating the mixture for a sufficient amount of time at a sufficient temperature and under reduced pressure below 1 atm with sufficient moisture so that a Maillard reaction occurs between the lipid amino groups and the reducing carbohydrate source sufficient to provide a Maillard reaction product coating on the micelles that is effective to prevent oxidation of the medicament; and
stopping the reaction before it proceeds significantly beyond the Amadori rearrangement steps of the Maillard reaction.

95. The method of claim 94, where said lipid-soluble medicament source comprises a lipid-soluble nutraceutical and/or a lipid-soluble pharmaceutical,
wherein the lipid-soluble nutraceutical is selected from the group consisting of basil oil, carrot seed oil, celery seed oil, citronella oil, coriander oil, garlic oil, grapefruit oil, lemon oil, marjoram oil, onion oil, organum oil, parsley oil, rosemary oil, sage oil, tarragon oil, thyme oil, cannabis oil, and mixtures of two or more thereof; and
wherein the lipid-soluble pharmaceutical is selected from the group consisting of chlortetracycline, oxytetracycline, demeclocycline, lymecycline, methacycline, minocycline, rolitetracycline, doxycycline, tigecycline, eravacycline, sarecycline, omadacycline, sulfacetamide, sulfadiazine, sulfadimidine, sulfafurazole (sulfisoxazole), sulfisomidine (sulfaisodimidine), sulfamethoxazole, sulfamoxole, sulfanitran, sulfadimethoxine, sulfamethoxypyridazine, sulfametoxydiazine, sulfadoxine, sulfametopyrazine, terephtyl, acetohexamide, carbutamide, chlorpropamide, glibenclamide (glyburide), glibornuride, gliclazide, glyclopyramide, glimepiride, glipizide, gliquidone, glisoxepide, tolazamide, tolbutamide, acetazolamide, bumetanide, chlorthalidone, clopamide, furosemide, hydrochlorothiazide, indapamide, mefruside, metolazone, xipamide, methazolamide, ethoxzolamide, sultiame, zonisamide, mafenide, amprenavir, darunavir, delavirdine, fosamprenavir, tipranavir, asunaprevir, beclabuvir, dasabuvir, grazoprevir, paritaprevir, simeprevir, azabon, apricoxib, celecoxib, parecoxib, bosentan, dronedarone, sotalol, tamsulosin, udenafil, dorzolamide, probenecid, sulfasalazine, sumatriptan, ciprofloxacin, garenoxacin, gatifloxacin, gemifloxacin, levofloxacin, moxifloxacin, azithromycin, clarithromycin, erythromycin, virginiomycine, clindamycin, lincomycin, pirlimycin, and mixtures of two or more thereof.

96. An emulsified lipid-soluble medicament composition, comprising micelles of lipid-soluble medicament encapsulated by a lipid emulsifying agent having at least one amino group, wherein the micelles are coated with the Maillard reaction product of said amino groups and a reducing sugar.
